(19) United States
(12) Patent Application Publication
(10) Pub. No.: US 2006/0230643 A1
(43) Pub. Date: Oct. 19, 2006

(54) Footwear with additional comfort

(76) Inventor: Michael Affleck, Aliso Viejo, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/387,096

(22) Filed: Mar. 22, 2006

Related U.S. Application Data
(60) Provisional application No. 60/664,358, filed on Mar. 23, 2005.

(51) Int. Cl. A61F 5/14

(52) U.S. Cl. 36/153; 36/154

(57) ABSTRACT

Disclosed is footwear, such as a sandal, having an outsole, an insole, and an upper. The upper is attached to the outsole and/or the insole and is meant to hold the foot in place when the sandal is worn. The outsole includes a bottom surface and a sidewall extending upwardly from the bottom surface to provide an area of insertion for the insole. The insole is comprised either partially or fully of a gel or gel-like substance and is placed within the gel insole receiving area of the outsole. The gel insole can be manufactured within the gel insole receiving area or manufactured separately and placed within the gel insole receiving area.

### CROSS-REFERENCE TO RELATED APPLICATIONS

This application claims the benefit of U.S. Provisional Application No. 60/664,358, filed Mar. 23, 2005, the entirety of which is hereby incorporated by reference.

### FIELD OF THE INVENTION

This invention relates generally to footwear, such as sandals, and specifically to footwear providing added comfort to the feet.

### Description of the Related Art

Athletes typically spend a lot of time on their feet. Certain sports, such as soccer, football, and baseball (to name a few) require that the participant wear cleats. At the end of a game, the athlete's feet are often sore, fatigued, and in need of rest.

A variety of materials are used to manufacture insoles available in sandals. U.S. Pat. No. 4,222,185 (Zeligman, et al.) describes an insole for summer footwear such as sandals, comprised of an insole molded from plastic material. U.S. Pat. No. 4,200,997 (Scheinhaus, et al.) describes a sandal having a multilayer sole including an insole of mini-cell foam. U.S. Pat. No. 4,275,512 describes a sandal with an upper insole section composed of leather and a lower insole section composed of composition rubber.

Special insoles for footwear are not uncommon and serve a variety of purposes. U.S. Pub. No. 2003/0136025 A1 (Galbraith, et al.) describes an insole for footwear, including a plurality of compressible protrusions. U.S. Pub. No. 2003/0005599 A1 (Panaccione) describes a modular cushioned insole support system.

Gel insoles have been used primarily in athletic shoes and orthopedic shoes to provide additional comfort or other desired benefits. U.S. Pat. No. 6,598,319 B2 (Hardt) describes an insole intended to provide cushion to the heel and/or the arch area of the foot for people who spend a lot of time on their feet. This particular insole has two openings that are filled with a polymeric gel composition. U.S. patent No. 2003/0024134 A1 (Howlett, et al.) provides another example of an insole that is partially composed of a viscoelastic gel.

### SUMMARY OF THE INVENTION

In some embodiments, the present invention is intended to provide the athlete's foot with some relief by introducing footwear with a comfortable insole comprise either partially or fully of a polymer gel or gel-like material that can be worn before or after the game. Such footwear can have an insole which is comprised either partially or fully of a gel or gel-like (hereinafter referred to simply as “gel”) substance. The gel insole is protected by an outsole comprised of a durable material as outlined in the body of this application. The gel insole, combined with the upper, provides a comfortable receiving area for the foot.

Some embodiments of the present invention relates to a sandal. The sandal includes an outsole, an insole, and an upper attached to the outside and/or the insole. The outsole includes a bottom wall and a sidewall extending from the bottom wall to provide a point of insertion for the insole. The insole is comprised either partially or fully of a gel or gel-like substance and is provided within the gel insole receiving area of the outside. The gel insole can be manufactured within the gel insole receiving area or manufactured separately and placed within the gel insole receiving area later in the manufacturing process. The upper is attached to the outsole and/or the insole and is meant (together with the insole) to provide a containment area for the foot. The upper can be made of one or more pieces. If the upper is made of more than one piece, the pieces can be removably combined with Velcro or some other material with similar utility such as snaps, zippers, buttons, or the like.

For athletes with tired or sore feet, some embodiments of the present invention provides a comfortable, soothing sandal to put on after a practice or game. Athletes who have their legs, ankles, or feet taped before a game will also benefit from such a sandal in that it provides a comfortable alternative to tight cleats or shoes that can cause discomfort and cramping before the game or match even starts.

In some embodiments, a sandal provides an insole that is comprised either partially or completely of a polymer gel material. This material will provide additional comfort to the foot by conforming to the foot and adding substantial padding to areas of the foot in need of rest and/or prone to fatigue and stress during exercise.

### BRIEF DESCRIPTION OF THE DRAWINGS

FIG. 1 is a perspective view of the outsole with the gel insole according to an embodiment of the invention;

FIG. 2 is a perspective view of an insole and an insole covering according to an embodiment of the invention;

FIG. 3 is a perspective view of a gel or gel-like substance, an insole, and an insole covering according to an embodiment of the invention;

FIG. 4 is a perspective view of a gel or gel-like substance, an insole with internal gates, and an insole covering according to an embodiment of the invention;

FIG. 5 shows individually packaged gel packets and a top plan view of an insole according to an embodiment of the invention. FIG. 5 also shows a covering for the individual gel packets;

FIG. 6 is a top plan view of an embodiment of a gel-based insole;

FIG. 7 is a top plan view of another embodiment of a gel-based insole;

FIG. 8 is a top plan view of another embodiment of a gel-based insole;

FIG. 9 is a top plan view of another embodiment of a gel-based insole;

FIG. 10 is a top plan view of another embodiment of a gel-based insole;

FIG. 11 is a top plan view of another embodiment of a gel-based insole;

FIG. 12 is a top plan view of another embodiment of a gel-based insole;

FIG. 13 is a top plan view of another embodiment of a gel-based insole;

FIG. 14 is a top plan view of an embodiment of an insole composed either partially or fully of gel or a gel-like substance;

FIG. 15 is a top plan view of an embodiment of an insole comprised of cloth, rubber, plastic, vinyl, foam, or some other material with pockets of gel at the arch of the foot, the ball of the foot, the heel of the foot, or some combination of the three, according to an embodiment of the invention;

FIG. 16 is a top plan view of an embodiment of an insole comprised of cloth, rubber, plastic, vinyl, foam, or some other material with pockets of gel provided in a uniform or random fashion throughout the insole according to an embodiment of the invention;

FIG. 17 is a perspective view of the outsole with the insole according to an embodiment of the invention;

FIG. 18 is a perspective view of the insole with a protective covering for the gel or gel-like substance as well as the outsole according to an embodiment of the invention;

FIG. 19 is a perspective view of the gel or gel-like substance, the outsole, the insole, and the insole cover according to an embodiment of the invention;

FIG. 20 is a perspective view of the outsole and the insole according to an embodiment of the invention; and

FIG. 21 is a perspective view of a sandal according to an embodiment of the invention.

### DETAILED DESCRIPTION OF THE PREFERRED EMBODIMENT

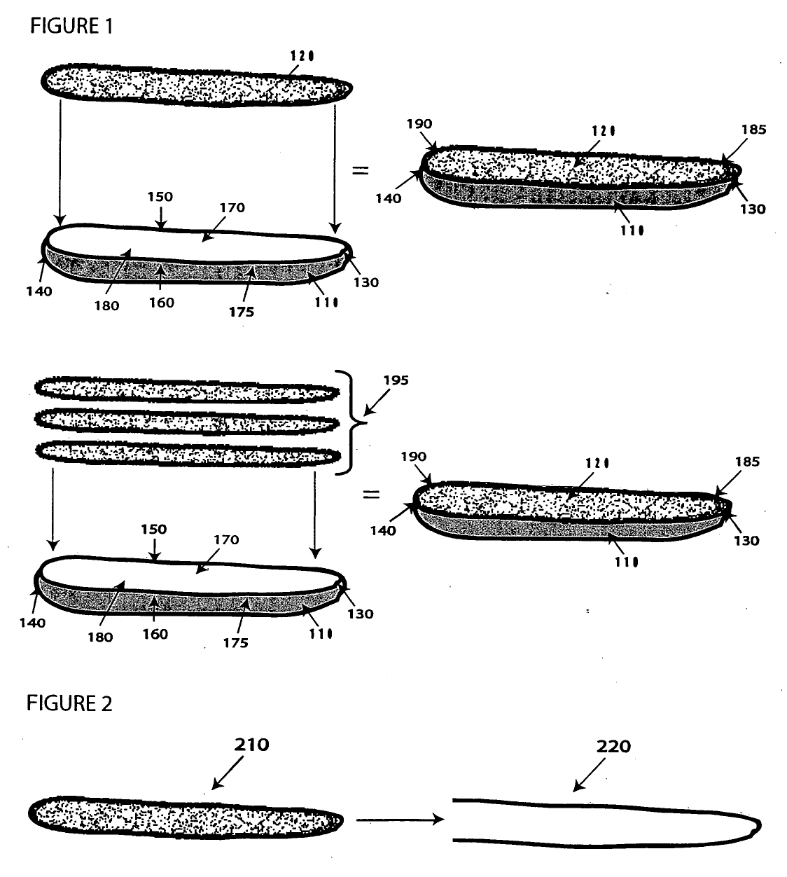

FIG. 1 is a representation of an outsole (110) and an insole (120). The outsole (110) has a front end (130) and a rear end (140); a left side (150) and a right side (160). The outsole (110) also has an inner wall (170), a perimeter wall (175), an inner bottom (180), and an outer bottom (not shown).

The outsole (110) can be composed of a polymer block copolymer, or other durable material. Examples of such material include SBS rubber, plastic, and foam to name a few. Such materials used in the outsole (110) provide durability, traction, support, and some cushion to the user of the sandal. In some embodiments, the material chosen for the outsole (110) can be chosen to withstand significant contact with rough ground surfaces such as concrete, dirt, grass, carpet, stone, wood, and asphalt.

The outsole (110) is generally formed such that the insole (120) is able to rest in the space formed by the combination of the inner wall (170) and inner bottom (180)—protected further by the perimeter wall (175) and outer bottom (not shown). This insole receiving area (170/180) can provide a protective environment into which a comfortable insole (120) may be manufactured directly or into which a comfortable insole (120) may be placed once it has been manufactured separately.

The insole (120) has a front end (185) and a rear end (190) and may be composed, for example, of a solid polymer gel, a viscoelastic polymer gel, a semi-liquid “squishy” polymer gel, or some combination of the above. The primary purpose of using a polymer gel material for the insole (120) is to provide comfort and relief to tired and sore feet. In some embodiments, the material used for the insole (120) is thick enough to provide outstanding comfort, yet thin enough to provide a high level of support. Throughout the remainder of this application, the insole will be referred to as a single layer of polymer gel or gel-like material (120); however, a multiple layer insole (190) could also be used.

As shown in FIG. 2, a solid or viscoelastic polymer gel/other solid material used to form the insole may be completely or partially exposed to the air (210) or contained in/covered with a cloth, rubber, nylon, plastic, vinyl, foam, or other type of material covering (220) to provide the insole with desired characteristics such as added comfort, traction, durability, protection, design, and/or styling.

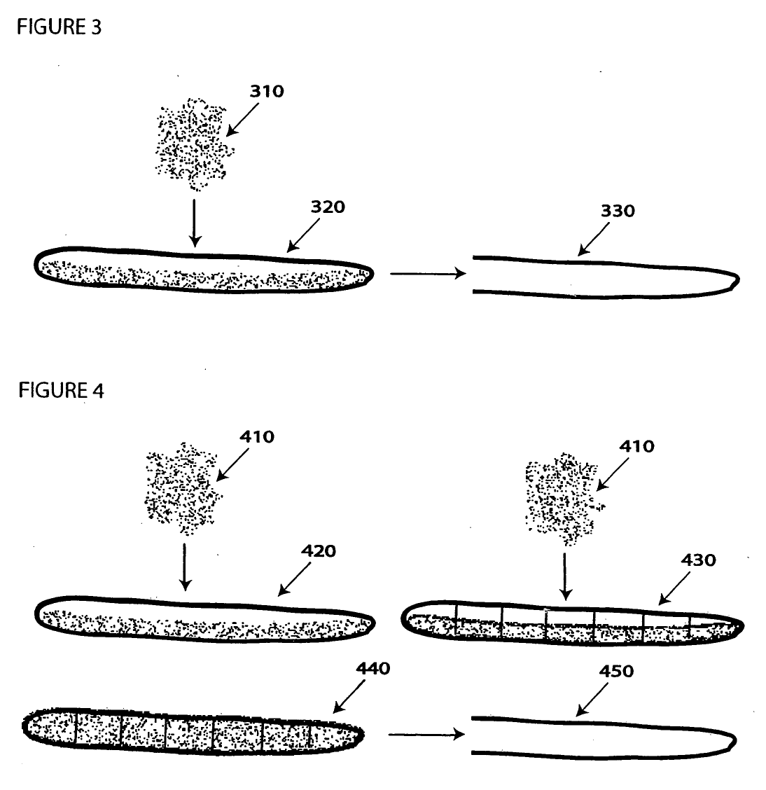

Referring to FIG. 3, a semi-liquid or “squishy” polymer gel (310) can be contained in some kind of container or pouch (320) to keep the material enveloped within the insole while the sandal is in use. This container (320) would preferably be composed of a plastic, rubber, or some other material capable of housing a liquid or semi-liquid substance without being prone to excessive damage which would allow the liquid to escape. For example, the material can be durable enough to hold the liquid without being ruptured or damaged by various amounts of pressure caused by the foot or other stress factors that could affect the semi-liquid or “squishy” polymer gel container (320). Also, as illustrated in FIG. 3, the container holding the semi-liquid or “squishy” polymer gel (320) could be completely exposed to the air (320) or contained in/covered with cloth, rubber, nylon, plastic vinyl, foam, or some other type of material covering (330). One purpose of the cover (330) is to provide desired characteristics such as added comfort, traction, durability, protection, design, and/or styling—similar to the application described for a solid or viscoelastic polymer gel application (see FIG. 2).

Referring to FIG. 4, a semi-liquid or “squishy” gel (410) may be enclosed in a one-piece pouch (420) or in a pouch with several internal gates or boundaries (430). Said gates could be formed vertically, horizontally, or in any number of directions or combination of directions. These gates or boundaries can be strategically placed within the pouch (420/430) to prevent excess amounts of gel from moving to any one area of the insole when pressure is applied to the pouch (420/430) under the weight of the user. For example, the gates can be placed so as to form several square inch enclosures, each of which would be filled with gel (440). In some embodiments, the gel can be free to move within its own square inch area, but not beyond. Thus, regardless of the amount of pressure being applied to the insole, the gel would maintain a relatively even distribution throughout the greater insole. As in other application, this conglomerate of pouches formed by the internal gates could be completely or partially exposed to the air (440) or contained in/covered with cloth, rubber, nylon, plastic, vinyl, foam, or some other type of material covering (450) to provide desired characteristics such as added comfort, traction, durability, protection, design, and/or styling.

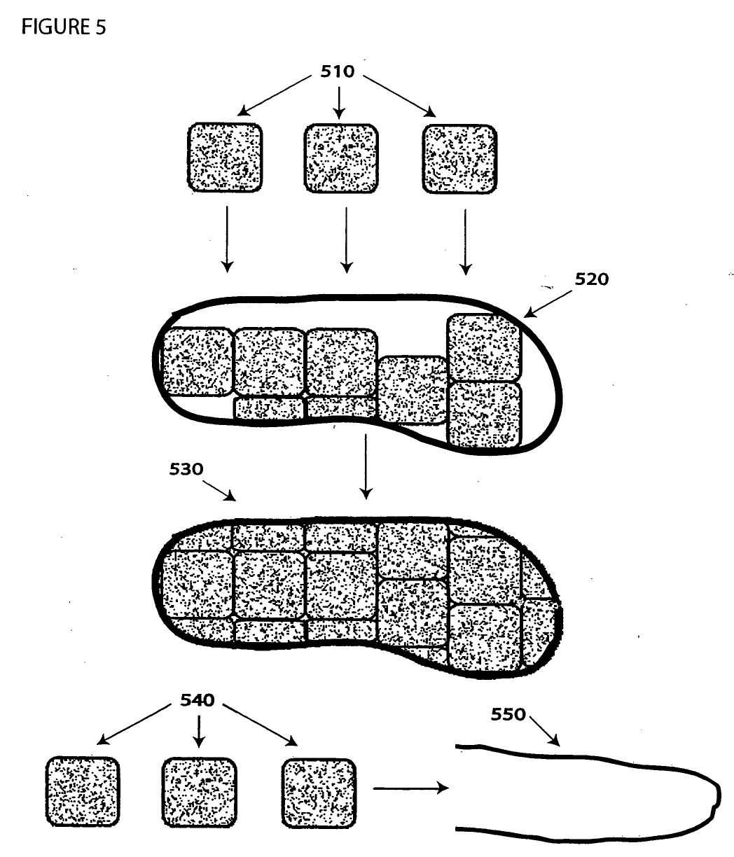

FIG. 5 provides a top plan illustration of an additional application in which the insole (520/530) could be produced using several separate gel pouches (510) placed side-by-side within the entire gel insole receiving area of the outsole (520) rather than being produced in a one-piece pouch that is separated by internal gates as shown in FIG. 4. This conglomerate of individual polymer gel packets could be packaged individually (540) and placed within the gel receiving area of the outsole (520), or packaged in a larger container or pouch (550) which would be placed in the gel receiving area of the outsole (520) for additional security, durability, and/or stability. As in applications described in FIGS. 2, 3, and 4, this conglomerate of pouches could be completely or partially exposed to the air (210) or contained in/covered with cloth, rubber, nylon, plastic, foam, or some other type of material covering (220) to provide desired characteristics such as added comfort, traction, durability, protection, design, and/or styling.

The insole (530) is not restricted to one specific type of polymer gel. Just as different parts of the foot have different sensitivities, a combination of solid or viscoelastic gel and semi-liquid or “squishy” polymer gel can be used in the insole (530). One purpose of using different materials for different areas of the foot is to provide additional comfort and support to specific areas of the foot prone to soreness, fatigue, or excessive irritation from blisters or other foot issues cased by cleats.

If several different types of gel are used, the “squishy” portion of the gel insole (530) can be contained in a special cloth, nylon, plastic, or rubber package as previously explained and shown in FIG. 4 (410/420). The package can then be integrated into the solid gel insole (530) base. The semi-liquid gel can be strategically placed in areas of the insole (530) that house portions of the foot requiring special attention.

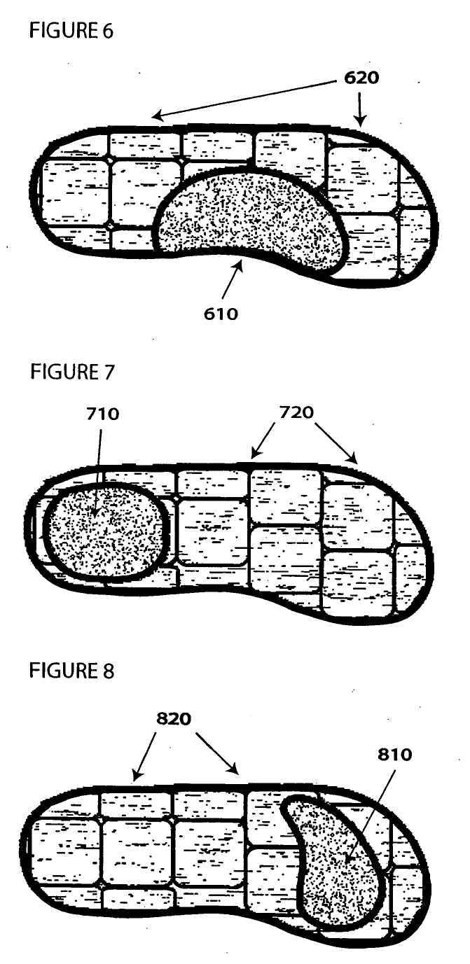

FIG. 6 shows one such area of the foot which, in some embodiments, can potentially require special attention—the arch (610). In this instance, the base of the insole can be comprised of a solid or viscoelastic polymer gel (620). A special semi-liquid or “squishy” polymer gel pocket can be assigned to the area of the insole that touches the arch of the foot (610).

FIG. 7 illustrates another potential area of sensitivity in the foot that could need special attention—the heel (710). In this instance, the base of the insole can be comprised of a solid or viscoelastic polymer gel (720). A special semi-liquid or “squishy” polymer gel pocket can be assigned to the area of the insole that touches the heel of the foot (710).

Likewise, the ball of the foot is an area that often causes athletes and other active people pain. As shown in FIG. 8, in this embodiment, the base of the insole can be comprised of a solid or viscoelastic polymer gel (820). A special semi-liquid or “squishy” polymer gel pocket can be assigned to the area of the insole that touches the ball of the foot (810).

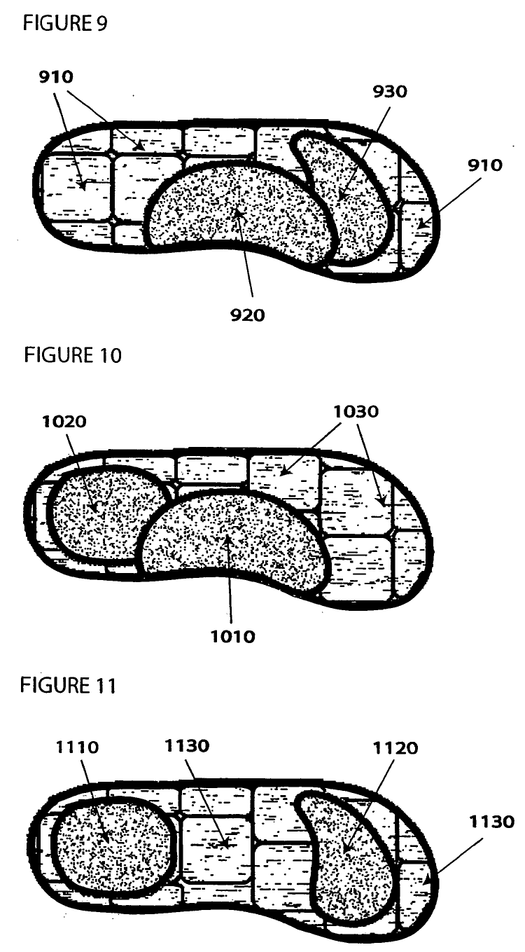

In addition to limiting the “squishy” polymer gel to one area of sensitivity as discussed in the previous three paragraphs, any combination of these three areas of sensitivity could be supplied with the semi-liquid or “squishy” gel. FIG. 9 gives one example. In this embodiment, the base of the insole could be comprised of a solid or viscoelastic polymer gel (910). The area of the insole that touches the arch (910) and the ball (930) of the foot can contain the semi-liquid or “squishy” polymer gel material.

Similarly, as shown in FIG. 10, a semi-liquid or “squishy” polymer gel can be applied to the arch of the foot (1010) and the heel of the foot (1020), both of which would benefit from the semi-liquid or “squishy” gel pockets. The rest of the insole can be comprised of a solid or viscoelastic polymer gel (1030).

FIG. 11 shows that the heel of the foot (1110) and the ball of the foot (1120) constitute another possible combination area for the semi-liquid or “squishy” gel pocket application. The remaining part of the insole can be comprised of the solid or viscoelastic polymer gel material (1130).

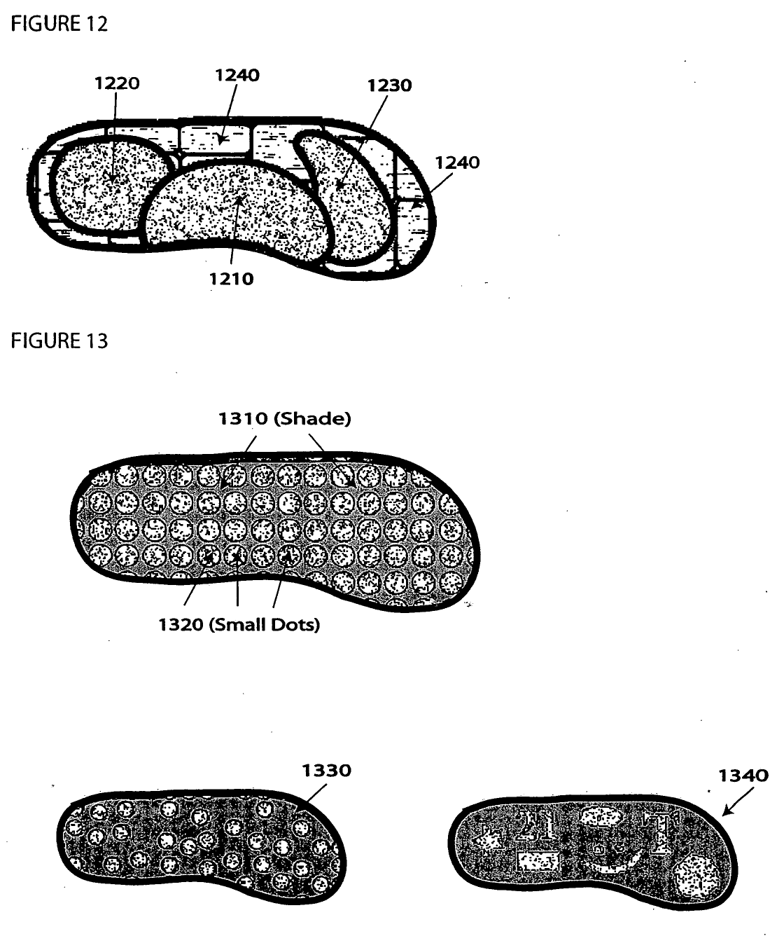

As illustrated schematically in FIG. 12, an insole covering all three of the previously mentioned areas of the foot; the arch (1210), the heel (1220), and the ball of the foot (1230), can be formed using the semi-liquid or “squishy” polymer gel material in one single application. The rest of the insole can be formed using a solid or viscoelastic polymer gel (1240).

Referring now to FIG. 13, a solid or viscoelastic polymer gel can be used for the base of the insole (1310) and the semi-liquid or “squishy” polymer gel can be applied in small pockets throughout the insole in a uniform manner (1320) to create additional comfort or other desired characteristics. These semi-liquid gel pockets can also be spread through the insole strategically or randomly to provide added comfort and to enhance the sandal's appearance and marketability through specific design cues (1330). For example, the gel pockets can be made to look like shapes, objects, letters, numbers, or any variety of possible pictures of symbols (1340).

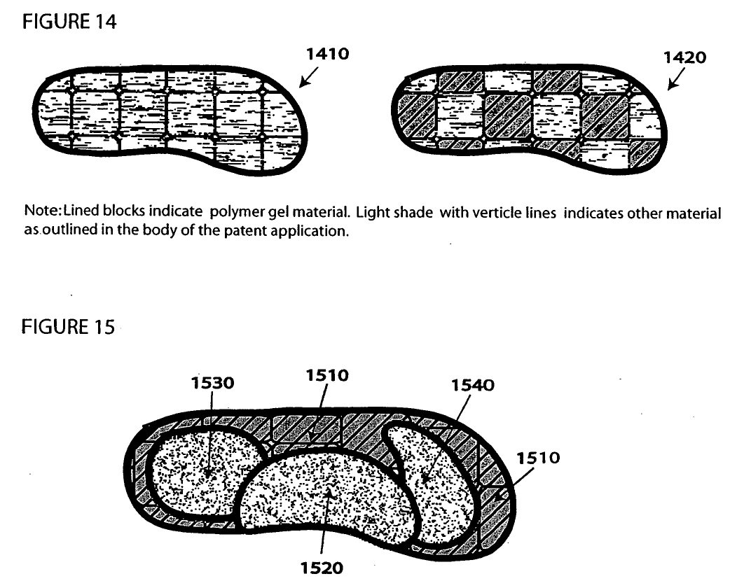

As illustrated in FIG. 14, in addition to an insole comprised completely of a polymer gel substance (1410), an insole comprised of a polymer gel, integrated or combined with another material such as cloth, rubber, plastic, or foam could also apply (1420). One purpose of a partially gel/partially rubber, cloth, foam, plastic, or other material insole (1420) is to add comfort, structure, stability, traction, durability, protection, support, and/or unique design to the sandal.

Similar to the solid gel base insole application previously discussed, several applications can be used with a base comprised of rubber, plastic, foam, or some other non-polymer gel material for the insole. For example, FIG. 15 shows how a polymer gel, solid or “squishy” (1520/30/40), can be strategically placed in or on areas of the rubber, plastic, foam, or other material base insole (1510) in such a way as to provide additional padding, massage, comfort, and relief from stress to the foot.

As seen in FIG. 15, one such area of the insole where gel can be applied within the rubber, plastic, foam, or other material base (1510) is area of the insole where the arch of the foot would rest (1520). Another possible area of the insole where the gel could be applied within the rubber, plastic, foam, or other material base insole (1510) is the part of the foot where the heel would rest (1530). Yet another area within the rubber, plastic, foam, or other material base insole (1510) where a solid or “squishy” polymer gel could be applied is the area of the insole where the ball of the foot would rest (1540).

As discussed earlier in this application, any combination of these three areas of the foot: the arch (1520), the heel (1530), and the ball (1540), can be used as areas where the gel would rest within the larger base insole made of such materials as plastic, rubber, foam, or some other materials (1510)—similar to the options show with the semisolid or solid viscoelastic polymer gel base in FIGS. 7-13.

For example, the area of the insole where the heel (1530) and the ball of the foot (1540) rest could contain a solid or semi-liquid “squishy” polymer gel with the rest of the insole being composed of plastic, rubber, foam, or some other material (1510). Likewise, the area of the insole where the arch (1520) and the heel (1530) rest can contain solid or “squishy” polymer gel with the rest of the insole being composed of plastic, rubber, foam, or some other material (1510). The area of the insole where the arch (1520) and the ball of the foot (1540) will rest can contain solid or “squishy” polymer gel with the rest of the insole being composed of the foot, the arch (1520), the heel (1530), and the ball (1540) can contain the solid or “squishy” polymer gel—placed within the larger base of the insole made of a plastic, rubber, foam, or some other durable material (1510). One purpose of the gel in these embodiments would be to provide additional comfort and support to specific areas of the foot prone to soreness, fatigues, or excessive irritation from blisters or other foot issues caused by cleats.

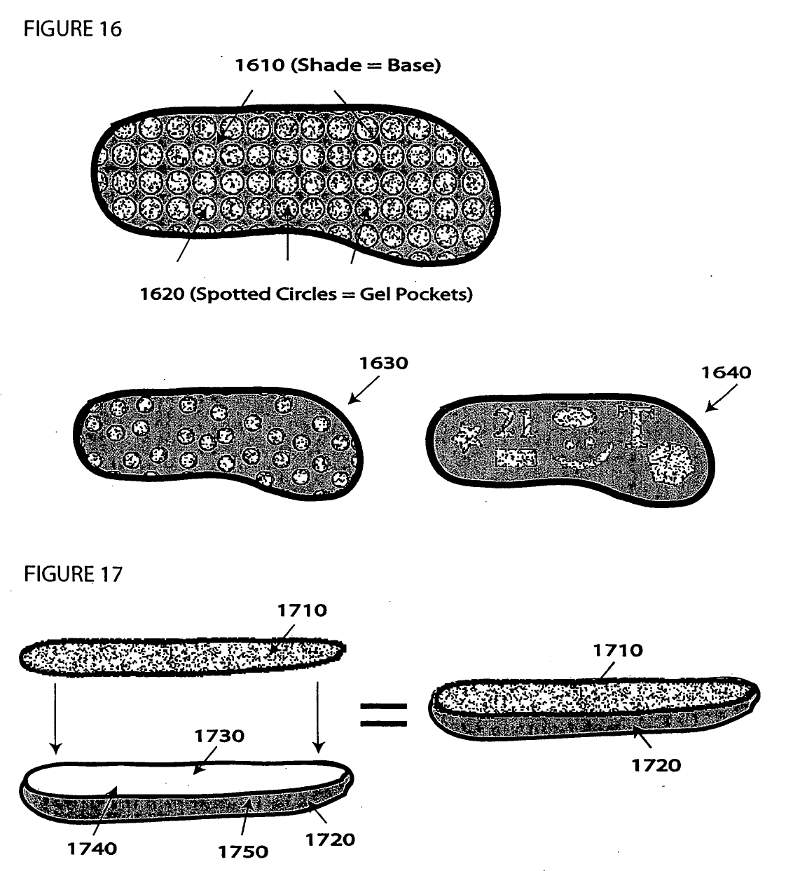

FIG. 16 illustrates another embodiment using an SBS rubber, plastic, foam, or some other material base (1610) combined with a solid or “squishy” polymer gel (1620). In this embodiment, the gel (1620) can be provided throughout the larger insole made of plastic, rubber, foam, or some other durable material (1610) in a uniform manner to create additional comfort or to enhance the sandal's appearance through specific design cues (1620).

FIG. 16 also shows that these gel pockets can be distributed strategically or randomly throughout the insole with the primary purpose of providing comfort and relaxation to specific pressure points located in the foot (1630). A strategic or random distribution of gel pockets within the SBS rubber, plastic, foam, or other material base insole (1610/20/30) can also be made with the dual purpose of providing desired design and styling as well as outstanding comfort.

Continuing with FIG. 16, many possible insole designs can achieve a strategically or randomly distributed gel applications. For example, the gel pockets can be scattered throughout the SBS rubber, plastic, foam, or other material base, and made to look like objects, shapes, letters, numbers, or any of a variety of possible pictures, or symbols (1640).

Once the material and design of the insole have been determined, at least four recommended manufacturing techniques can be used. Other manufacturing techniques known to those of skilled in this field could also be used.

Shown in FIG. 17, one of these methods involves the gel insole (1710) manufactured as a separate (to the outsole (1720)) piece and subsequently placed in the insole receiving area (1730/1740) created by the inner side wall (1730) and inner bottom (1740) of the outsole (1720). This independent (from the outsole (1720)) insole (1710) can then be sewn to the perimeter wall (1750) and/or bottom (not shown) of the outsole (1720) or attached to the inner side wall (1730) and/or inner bottom (1740) of the outsole (17200) with an adhesive substance such as glue. This method of manufacturing can be carried out by a single entity or split into two or more entities.

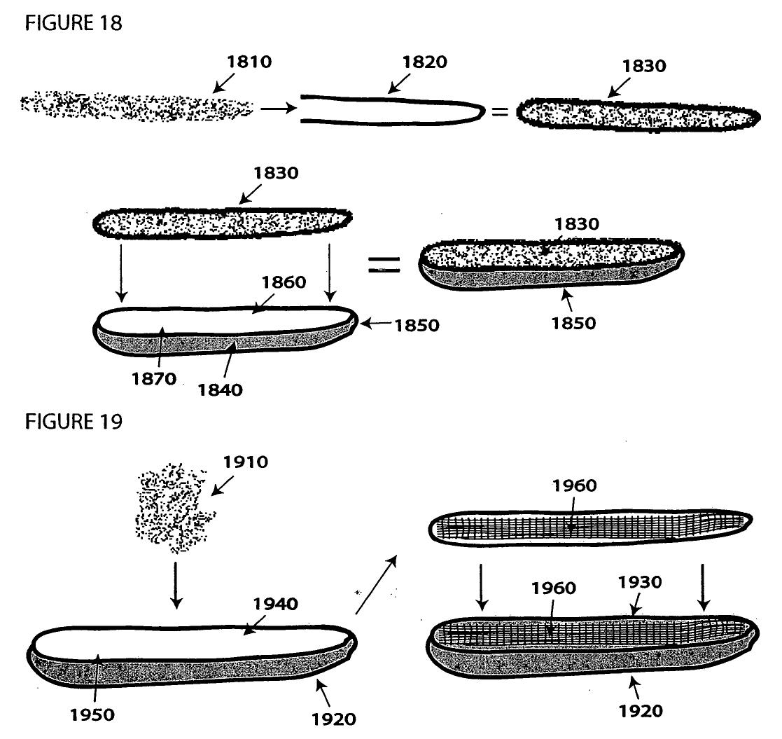

FIG. 18 shows another manufacturing technique that can be applied to the sandal, protective material (1820) into which the insole (1810) can be placed. Once combined, this newly created insole (1830) could then be sewn to the perimeter wall (1840) and/or bottom (not shown) of the outsole (1850) or attached to the inner side wall (1860) and/or inner bottom (1870) with an adhesive substance such as glue.

FIG. 19 shows that if a semi-liquid or “squishy” polymer gel (1910) is chosen for a portion (or all) of the insole (1930), the area formed by the inner side wall (1940) and inner bottom (1950) of the outsole (1920) can act as a receptacle for this gel, thus forming an insole (1930) integrated into the outsole (1920). In this instance, the gel (1920) can be placed directly into the outsole receiving area (1940/1950) and then be covered by a plastic, rubber, vinyl, or some other suitable, durable material or combination of materials (1960). This covering (1960) can protect the gel and prevent it from seeping out of the gel receiving area (1940/1950) of the outsole (1920). As mentioned earlier in this application, a semi-liquid or “squishy” gel insole (1930) solution would likely include gates or boundaries so the gel would not move significantly to any one area when pressure from the foot or body is applied to the insole (see FIGS. 4 and 5).

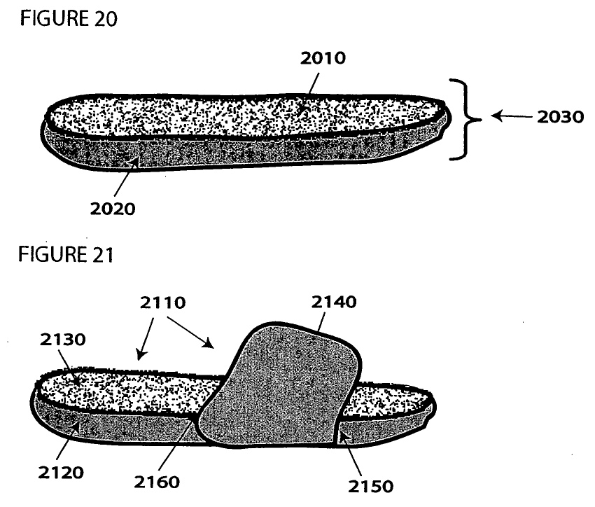

As shown in FIG. 20, the polymer gel insole (2010) can be simply manufactured as one piece within the SBS rubber, plastic, vinyl, or other material base outsole (2020). This solution would probably work best with a partial gel-based insole (2010) rather than with a completely gel base insole (2010)—especially if a “squishy” polymer gel is being used for a portion of the insole (2010). Also, a solid or viscoelastic gel insole (2010) would likely be easier than a semi-liquid or “squishy” gel insole (2010) to manufacture as part of the complete combined sole of the sandal.

FIG. 21 illustrates an embodiment of a sandal (2110) according to the invention. The sandal (2110) is comprised of an outsole (2120), an insole (2130), and an upper (2140). The upper (2140) is preferably attached to the perimeter wall (2150) of the outsole (2120) on both sides. The upper (2140) can be sewn, glued, or otherwise attached to the inner side wall (2160) and/or perimeter wall (2150) of the outsole (2120). The upper (2140) can be constructed of one or more pieces depending on the desired functionality of the sandal. A one-piece upper (2140) can provide more stability and security to the foot. A two-piece (or more) upper (2140) can provide a tighter, more customized fit to the individual foot. The upper (2140) can be comprised of any material deemed sturdy enough to support the foot during use. The upper (2140), combined with the insole (2130), provides a receiving area for the foot. The upper (2140), combined with the insole (2130), provides a receiving area for the foot. The upper (2140) is intended to help keep the foot in place while the sandal is in use.

What is claimed is:

1. A sandal comprising:
a durable outsole with an upper surface and a bottom surface;
a compliant pouch permanently attached to the upper surface of the outsole;
a gel material disposed within the compliant pouchs; and
an upper attached to the outsole configured to receive a user's foot and to assist in retaining the foot against the compliant pouch.

2. The sandal of claim 1, further comprising a plurality of compliant pouches filled with a gel material;

3. The sandal of claim 1, further comprising a plurality of different regions on or near the upper surface of the outsole, wherein at least two of the regions have different degrees of hardness.

4. The sandal of claim 3, wherein at least one of the regions is configured to contact a user's heal during use.

5. The sandal of claim 3, wherein at least one of the regions is configured to contact a user's foot arch during use.

6. The sandal of claim 3, wherein at least one of the regions is configured to contact a user's ball of the foot during use.

7. The sandal of claim 1, wherein the pouch covers substantially the entire upper surface of the outsole.

8. The sandal of claim 1, wherein the pouch includes partitions to inhibit the movement of the gel material within the pouch.

9. The sandal of claim 1, wherein the gel material comprises polymer gel.

10. The sandal of claim 1, wherein the pouch comprises a material selected from the group consisting of cloth, rubber and nylon.

11. The sandal of claim 1, wherein the pouch comprises a material selected from the group consisting of plastic, vinyl, and foam.
